(19) United States
(12) Patent Application Publication
(10) Pub. No.: US 2024/0285618 A1
(43) Pub. Date: Aug. 29, 2024

(54) PHARMACEUTICAL COMPOSITIONS OF LUMATEPERONE

(71) Applicants: Saravanan Kannusamy, Hyderabad (IN); Prabhakaran Chakkirala, Hyderabad (IN); Jagadeesh Gorsa, Hyderabad (IN); Nagaprasad Vishnubhotla, Hyderabad (IN); Sivakumaran Meenakshisunderam, Hyderabad (IN)

(72) Inventors: Saravanan Kannusamy, Hyderabad (IN); Prabhakaran Chakkirala, Hyderabad (IN); Jagadeesh Gorsa, Hyderabad (IN); Nagaprasad Vishnubhotla, Hyderabad (IN); Sivakumaran Meenakshisunderam, Hyderabad (IN)

(21) Appl. No.: 18/581,632

(22) Filed: Feb. 20, 2024

(30) Foreign Application Priority Data
Feb. 20, 2023 (IN) 202341011585

(51) Int. Cl. A61K 31/4985; A61K 9/00; A61K 9/48

(52) U.S. Cl. CPC A61K 31/4985; A61K 9/0053; A61K 9/4816; A61K 9/4833; A61K 9/4858; A61K 9/4866

(57) ABSTRACT

The present disclosure relates to pharmaceutical composition comprising Lumateperone or pharmaceutically acceptable salt form and, processes for manufacture thereof.

### CROSS REFERENCE TO RELATED APPLICATION

This application claims priority from an Indian Patent Application IN 202341011585 filed on Feb. 20, 2023.

### FIELD OF THE INVENTION

The present disclosure relates to pharmaceutical composition comprising Lumateperone or pharmaceutically acceptable salt form and processes for manufacture thereof.

### BACKGROUND OF THE INVENTION

Lumateperone tosylate is chemically known as 4-((6bR,10aS)-3-methyl-2,3,6b,9,10,10a-hexahydro-1H,7H-pyrido[3′,4′:4,5]pyrrolo[1,2,3-de]quinoxalin-8-yl)-1-(4-fluoro-phenyl)-butan-1-one 4-methylbenzenesulfonate. Its molecular formula is C₃₁H₃₆FN₃O₄S and its molecular weight is 565.71 g/mol and is represented by the following formula:

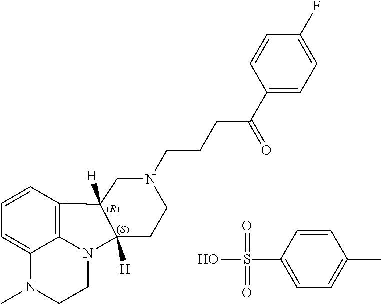

Lumateperone is commercially available under the brand name CAPLYTA® in Eq 10.5 mg Base, Eq 21 mg Base and Eq 42 mg Base capsules and marketed by Intra-Cellular Inc. in the United States for the treatment of Schizophrenia in adults and depressive episodes associated with bipolar I or II disorder (bipolar depression) in adults, as monotherapy and as adjunctive therapy with lithium or valproate.

Lumateperone and its acceptable pharmaceutical salts are described in U.S. Pat. No. RE39,680; U.S. Pat. No. 8,648,077 describes polymorphs A and B of tosylate salt of Lumateperone.

U.S. Pat. No. 9,616,061 discloses Lumateperone and pharmaceutical composition in oral unit dose form comprising an amount of from 0.1-20 mg in combination or association with a pharmaceutically acceptable diluent or carrier, provided that in the case of a salt, the weight is calculated as the free base.

U.S. Pat. No. 10,695,345 discloses a pharmaceutical capsule for oral administration, comprising Lumateperone in mono-tosylate salt form, wherein the Lumateperone mono-tosylate is in solid crystal form; and wherein the capsule comprises a blend of 10 to 30% by weight of Lumateperone mono-tosylate in solid crystal form, 60 to 90% by weight of mannitol, 0.5 to 10% by weight of croscarmellose sodium, 0.1 to 1% by weight of talc, and 0.1 to 3% by weight of magnesium stearate, filled into a gelatin capsule.

U.S. Pat. No. 11,052,084 discloses a pharmaceutical capsule for oral administration, comprising Lumateperone in mono-tosylate salt form, wherein the Lumateperone mono-tosylate is in solid crystal form; and wherein the capsule comprises a blend of 10 to 30% by weight of Lumateperone mono-tosylate in solid crystal form, 60 to 90% by weight of mannitol, 0.5 to 10% by weight of croscarmellose sodium, 0.1 to 1% by weight of talc, and 0.1 to 3% by weight of magnesium stearate, filled into a gelatin capsule, wherein the capsule comprises the Lumateperone mono-tosylate in an amount equivalent to 0.01 to 30 mg of Lumateperone free base.

U.S. Patent Publication No. 20210220280 covers a solid dosage form comprising Lumateperone in free or a salt form wherein the dosage form can be an immediate release dosage form and discloses mannitol as diluent.

The above prior arts disclose mannitol as the diluent for preparing pharmaceutical compositions Lumateperone and also having disintegrating agent. The inventors of the present invention have surprisingly found that the use of mannitol requires high amount of lubricant to reduce processing problems and requires high amount of disintegrating agent to get disintegration time.

In view of above, there is a need to develop a simple, reproducible, cost effective compositions of Lumateperone or its derivatives which offers desired pharmaceutical attributes such as dissolution, stability, bioavailable and manufactured by simple, reproducible, and commercially viable process at industrial scale. The present inventors have developed a solid oral pharmaceutical composition of Lumateperone or pharmaceutically acceptable salts thereof which is free of mannitol and/or disintegrating agent and the said composition exhibits adequate drug dissolution profile.

### SUMMARY OF THE INVENTION

The present invention provides a solid oral pharmaceutical composition comprising Lumateperone or a pharmaceutically acceptable salt thereof and one or more pharmaceutically acceptable excipients, wherein the composition is free of mannitol.

In one aspect of the present invention, it provides a solid oral pharmaceutical composition comprising: a) Lumateperone or a pharmaceutically acceptable salt thereof; b) one or more diluents; wherein weight ratio of Lumateperone to diluent is from about 1:2 to about 1:25; wherein the composition is free of mannitol.

In one aspect of the present invention, it provides a solid oral pharmaceutical composition comprising Lumateperone or a pharmaceutically acceptable salt thereof and one or more pharmaceutically acceptable excipients, wherein the composition is free of disintegrating agent.

In one aspect of the present invention, it provides a capsule composition comprising: a) Lumateperone or a pharmaceutically acceptable salt thereof; b) one or more diluents; wherein weight ratio of Lumateperone to diluent is from about 1:2 to about 1:25; wherein the composition is free of mannitol and/or disintegrating agent.

In another aspect of the present invention, it provides a process of making a capsule composition a) Lumateperone or a pharmaceutically acceptable salt thereof; b) one or more diluents; wherein weight ratio of Lumateperone to diluent is from about 1:2 to about 1:25; wherein the composition is free of mannitol and/or disintegrating agent.

In another aspect of the present invention, it provides a process of making a capsule composition a) Lumateperone or a pharmaceutically acceptable salt thereof; b) one or more diluents; wherein weight ratio of Lumateperone to diluent is from about 1:2 to about 1:10; wherein the composition is free of mannitol and disintegrating agent.

In another aspect of the present invention, it provides a capsule composition comprising:
a) 1% to about 50% by weight of Lumateperone or a pharmaceutically acceptable salt;
b) about 20% to about 95% by weight of diluents;
wherein the composition is free of mannitol and disintegrating agent and not less than 80% of Lumateperone dissolves within 30 minutes in a 500 ml of 0.1N Hydrochloric acid at a temperature of 37 ±0.5° C. using a USP apparatus-2 (paddle) with sinker at a rotation of about 50 rpm.

In one aspect of the present invention, it provides a capsule composition comprising: a) Lumateperone or a pharmaceutically acceptable salt thereof; b) one or more diluents; wherein weight ratio of Lumateperone to diluent is from about 1:2 to about 1:25; wherein the composition is free of mannitol and disintegrating agent and the composition is filled into the hydroxypropyl methylcellulose capsule shell.

In yet another aspect of the present invention it provides use of a solid oral pharmaceutical composition of the present invention in the manufacture of a medicament for treating Schizophrenia in adults and depressive episodes associated with bipolar I or II disorder (bipolar depression) in adults, as monotherapy and as adjunctive therapy with lithium or valproate.

### DETAILED DESCRIPTION OF THE INVENTION

The present invention provides a solid oral pharmaceutical composition comprising Lumateperone, in free, or pharmaceutically acceptable salt form, free of mannitol and/or a disintegrating agent, processes for manufacture thereof and methods of use in the treatment or prophylaxis of disease.

The present invention provides a capsule composition comprising Lumateperone, in free, or pharmaceutically acceptable salt form, free of mannitol and disintegrating agent, processes for manufacture thereof and methods of use in the treatment or prophylaxis of disease.

The term “Lumateperone” as used in the context of the present specification includes “Lumateperone” per se or its pharmaceutically acceptable salts, esters, derivatives, solvates, hydrates, enantiomers, isomers, polymorphs, prodrugs in thereof. The term “pharmaceutically acceptable salt” refers to salts derived from a variety of organic and inorganic counter ions including fumarate, maleate, phosphate, tosylate, L-tartrate, citrate, acetate, oxalate and sulfate. According to the present invention, Lumateperone tosylate is being preferred.

According to the present disclosure, Lumateperone is present in an amount from 0.1% to about 80% by weight based on the total weight of the composition, preferably Lumateperone is present in the form of Lumateperone tosylate in an amount from 0.1% to about 50% by weight based on the total weight of the composition. Lumateperone tosylate may be present in crystalline or amorphous form. U.S. Pat. Nos. 8,648,077; 9,199,995 and 9,586,960 taught several solid crystalline polymorphs of Lumateperone tosylate.

The term “formulation” or “composition” is used in the present specification interchangeably, and it includes solid oral pharmaceutical composition, is intended to encompass a drug product comprising Lumateperone or its pharmaceutically acceptable salts thereof, and other inert ingredient(s) or pharmaceutically acceptable excipients. Pharmaceutical composition of the invention include, but is not limited to, granules, tablets, immediate release tablets, caplets, capsules (hard or soft gelatin capsules, non-gelatin capsule), pellets, multiparticulate composition and the like. Preferably, the pharmaceutical composition refers to tablets and capsules. More preferably, the pharmaceutical composition refers to immediate release oral capsules, which may be uncoated or coated and capsule shells are made up of cellulose or gelatin, preferably cellulose based, more preferably hydroxypropyl methylcellulose based.

The term “pharmaceutically acceptable excipient”, means a pharmacologically inactive component such as a diluent or filler, binder, disintegrant, glidant, lubricant, coloring agent or the like. The excipients that are useful in preparing a pharmaceutical composition are generally safe, non-toxic and are acceptable for veterinary as well as human pharmaceutical use.

The term “diluent” or “filler” as used herein is defined as an excipient designed to increase the weight and/or the size of the pharmaceutical composition. Sometimes referred to as fillers, diluents often comprise a significant proportion of the dosage form, and the quantity and type of diluent selected often depends on its physical and chemical properties. Fillers fill out the size of a tablet or capsule, making it practical to produce and convenient for the consumer to use. By increasing the bulk volume, the fillers make it possible for the final product to have the proper volume for patient handling. Good filler must be inert, compatible with the other components of the formulation, non-hygroscopic, relatively cheap, compactible, and preferably tasteless or pleasant tasting.

Examples of diluents include, but are not limited to microcrystalline cellulose, sodium alginate, silicified microcrystalline cellulose, microfine cellulose, maltitol, lactose, anhydrous lactose, lactose monohydrate, spray dried lactose, maltodextrin, isomalt, dicalcium phosphate, dibasic calcium phosphate, tribasic calcium phosphate, sucrose, dextrose, magnesium carbonate and mixtures thereof. Diluent is present according to the present invention in an amount from about 5% to about 99%. Preferably, the amount of diluent is from about 10% to about 95% w/w, more preferably, the amount of diluent is from about 20% to about 90% w/w. Preferred diluent according to the present invention is selected from sucrose, microcrystalline cellulose or a combination thereof.

In one of the embodiments of the present invention it provides a solid oral pharmaceutical composition comprising: a) Lumateperone or a pharmaceutically acceptable salt thereof; b) one or more diluents selected from microcrystalline cellulose, sucrose, or combinations thereof; wherein weight ratio of Lumateperone to diluent is from about 1:2 to about 1:25; wherein the composition is free of mannitol.

In one of the embodiments of the present invention, it provides a capsule composition comprising: a) Lumateperone or a pharmaceutically acceptable salt thereof; b) one or more diluents; wherein weight ratio of Lumateperone to diluent is from about 1:2 to about 1:25; wherein the composition is free of mannitol and disintegrating agent.

In one of the embodiments of the present invention, it provides a capsule composition comprising: a) Lumateperone or a pharmaceutically acceptable salt thereof; b) one or more diluents; wherein weight ratio of Lumateperone to diluent is from about 1:2 to about 1:25; wherein the composition is free of mannitol and disintegrating agent and the composition is filled into the cellulose capsule shells.

The solid oral pharmaceutical composition of the invention may be prepared by conventional processes known to those of ordinary skill in the art, including, but not limited to, wet granulation, dry granulation such as slugging or compaction, or direct compression of the formulation into tablets or filling into capsules.

Examples of binders include, but not limited to group comprising of pregelatinized starch; partially pregelatinized starch; cellulose or a derivative thereof such as microcrystalline cellulose, methyl cellulose, ethyl cellulose, sodium carboxymethyl cellulose, hydroxyethyl cellulose, hydroxyethyl methyl cellulose, hydroxypropyl methyl cellulose and mixture thereof. The binder according to present invention may be present in an amount from about 0.5% to about 5.0% by weight with respect to total weight of the pharmaceutical composition, preferably, 1% to 4% or 2% or 2.1% or 2.2% or 2.5% or 3% w/w.

The term “lubricant” as used herein is defined as an agent to be incorporated into formulations to reduce the frictional forces between particles and between particles and metal contact surfaces of manufacturing equipment such as tablet punches and dies used in the manufacture of solid dosage forms. The lubricant may be present in the pharmaceutical composition in the form of a single compound or in the form of a mixture of compounds.

Examples of lubricants include, but are not limited to stearic acid, zinc stearate, sodium stearyl fumarate, magnesium stearate, sucrose stearate, aluminum stearate, adipic acid, carnauba wax, glycerol ester of fatty acid, calcium stearate, glycerol tribehenate, glyceryl behenate, polyethylene glycol and mixtures thereof. Lubricant is present according to the present invention in an amount from about 0.1% to about 3% w/w, preferably, the amount of lubricant is from about 0.25% to about 2.75% w/w, more preferably, the amount of lubricant is from about 0.5% to about 2.5% w/w. Preferred lubricant according to the present invention is magnesium stearate.

The term “glidant” as used herein is defined refers to substances used in tablet and capsule formulations to improve flow-properties to produce an anti-caking effect. Examples of glidants include, but are not limited to talc, colloidal silica, calcium silicate, magnesium silicate, magnesium trisilicate, fumed silica, bentonite, magnesium carbonate, glyceryl monostearate, glyceryl palmitostearate, light anhydrous silicic acid, crystalline cellulose, calcium phosphate tribasic and a combination thereof. Glidant is present according to the present invention in an amount from about 0.1% to about 3% w/w, preferably, the amount of lubricant is from about 0.25% to about 2.75% w/w, more preferably, the amount of lubricant is from about 0.1% to about 2.5% w/w. Preferred glidant according to the present invention is talc.

Examples of disintegrating agents used in various pharmaceutical compositions include, but are not limited to crospovidone, sodium starch glycolate, croscarmellose sodium, carboxymethyl cellulose sodium, polacrilin potassium and mixtures thereof.

The term “free of mannitol” according to the present invention means the composition is substantially free of mannitol. The term “free of disintegrating agents” according to the present invention means the composition is substantially free of disintegrating agents.

The term “free of mannitol and/or disintegrating agents” according to the present invention means the composition is substantially free of mannitol and disintegrating agents or the composition is substantially free of either mannitol or disintegrating agents.

In one of the embodiments of the present invention it provides a solid oral pharmaceutical composition comprising: a) Lumateperone or a pharmaceutically acceptable salt thereof; b) one or more diluents selected from microcrystalline cellulose, sucrose, or combinations thereof; wherein weight ratio of Lumateperone to diluent is from about 1:2 to about 1:25; wherein the composition is free of mannitol and disintegrating agent.

In one of the embodiments of the present invention it provides a capsule composition comprising: a) Lumateperone or a pharmaceutically acceptable salt thereof; b) microcrystalline cellulose as diluent; c) talc as glidant; and d) magnesium stearate as lubricant; wherein the composition is free of mannitol and disintegrating agent.

In one of the particular embodiments of the present invention it provides a capsule composition comprising:
a) 1% to about 50% by weight of Lumateperone or a pharmaceutically acceptable salt;
b) about 20% to about 95% by weight of diluents selected from microcrystalline cellulose, sucrose, or combinations thereof;
wherein the composition is free of mannitol and disintegrating agent.

In one of the embodiments of the present invention it provides a process of making a solid oral pharmaceutical composition comprising steps of:
a) co-sifting Lumateperone tosylate with diluent;
b) sifting glidant separately;
c) co-sifting materials of step a) & b);
d) sifting lubricant separately;
e) blending material of step c) in low shear blender;
f) blending & lubricating material of step e) with material of step d);
g) filling the lubricated blend of step f) into the cellulose based capsule of suitable size.

In one of the embodiments of the present invention it provides a capsule composition comprising:
a) 1% to about 50% by weight of Lumateperone or a pharmaceutically acceptable salt;
b) about 20% to about 95% by weight of diluents selected from microcrystalline cellulose, sucrose, or combinations thereof;
wherein the composition is free of mannitol and disintegrating agent and not less than 80% of Lumateperone dissolves within 30 minutes in a 500 ml of 0.1N Hydrochloric acid at a temperature of 37 ±0.5° C. using a USP apparatus-2 (paddle) with sinker at a rotation of about 50 rpm.

In yet another embodiments of the present invention it provides use of a solid oral pharmaceutical composition of the present invention in the manufacture of a medicament for treating Schizophrenia in adults and depressive episodes associated with bipolar I or II disorder (bipolar depression) in adults, as monotherapy and as adjunctive therapy with lithium or valproate.

The term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### Example 1

| Ingredients | % w/w |
|---|---|
| Lumateperone* | 20 |
| Microcrystalline cellulose | 65 |
| Lactose anhydrous | 8.7 |
| Sodium starch glycolate | 5 |
| Colloidal silicon dioxide | 0.3 |
| Sodium stearyl fumarate | 1 |
| Total | 100 |

*Lumateperone equivalent to Lumateperone tosylate

### Brief Manufacturing Process:

a) microcrystalline cellulose, lactose anhydrous, sodium starch glycolate, and colloidal silicon dioxide were co-sifted through appropriate screens;
b) sodium stearyl fumarate was separately sifted through appropriate screen;
c) Lumateperone and materials of step a) & some portion of step b) were blended in low shear blender;
d) blended material of step c) were lubricated with the remaining portion of material of step b);
e) the final lubricated blend of step d) were filled into the cellulose based capsule shell of suitable size.

### Example 2

| Ingredients | % w/w |
|---|---|
| Lumateperone* | 20 |
| Isomalt | 71 |
| Croscarmellose sodium | 7 |
| Colloidal silicon dioxide | 0.5 |
| Magnesium stearate | 1.5 |
| Total | 100 |

*Lumateperone equivalent to Lumateperone tosylate

### Brief Manufacturing Process:

a) isomalt, croscarmellose sodium and colloidal silicon dioxide were co-sifted through appropriate screens;
b) magnesium stearate was separately sifted through appropriate screen;
c) Lumateperone and materials of step a) & some portion of step b) were blended in low shear blender,
d) blended material of step c) were lubricated with the remaining portion of material of step b);
e) the final lubricated blend of step d) were filled into the cellulose based capsule shell of suitable size.

### Example 3

| Ingredients | % w/w |
|---|---|
| Lumateperone* | 20 |
| Maltitol | 72.9 |
| Croscarmellose sodium | 5 |
| Colloidal silicon dioxide | 0.6 |
| Magnesium stearate | 1.5 |
| Total | 100 |

*Lumateperone equivalent to Lumateperone tosylate

### Brief Manufacturing Process:

a) maltitol, croscarmellose sodium and colloidal silicon dioxide were co-sifted through appropriate screens;
b) magnesium stearate was separately sifted through appropriate screen;
c) Lumateperone and materials of step a) & some portion of step b) were blended in low shear blender;
d) blended material of step c) were lubricated with the remaining portion of material of step b);
e) the final lubricated blend of step d) were filled into the cellulose based capsule shell of suitable size.

### Example 4

| Ingredients | % w/w |
|---|---|
| Lumateperone* | 20 |
| Isomalt | 18 |
| Lactose anhydrous | 55 |
| Sodium starch glycolate | 5 |
| Colloidal silicon dioxide | 0.3 |
| Magnesium stearate | 1.7 |
| Total | 100 |

*Lumateperone equivalent to Lumateperone tosylate

### Brief Manufacturing Process:

a) isomalt, lactose anhydrous, sodium starch glycolate and colloidal silicon dioxide were co-sifted through appropriate screens;
b) magnesium stearate was separately sifted through appropriate screen;
c) Lumateperone and materials of step a) & some portion of step b) were blended in low shear blender;
d) blended material of step c) were lubricated with the remaining portion of material of step b);
e) the final lubricated blend of step d) were filled into the gelatin based capsule shell of suitable size.

### Example 5

| Ingredients | % w/w |
|---|---|
| Lumateperone* | 5 |
| Sucrose | 43 |
| Microcrystalline cellulose | 45 |
| Croscarmellose sodium | 5 |
| Talc | 0.5 |
| Magnesium stearate | 1.5 |
| Total | 100 |

*Lumateperone equivalent to Lumateperone tosylate

### Brief Manufacturing Process:

a) sucrose, microcrystalline cellulose, croscarmellose sodium and talc were co-sifted through appropriate screens;
b) magnesium stearate was separately sifted through appropriate screen;
c) Lumateperone and materials of step a) & some portion of step b) were blended in low shear blender;
d) blended material of step c) were lubricated with the remaining portion of material of step b);
e) the final lubricated blend of step d) were filled into the cellulose based capsule shell of suitable size.

### Example 6

| Ingredients | % w/w |
|---|---|
| Lumateperone* | 10 |
| Sucrose | 68.5 |
| Microcrystalline cellulose | 20 |
| Talc | 0.5 |
| Magnesium stearate | 1 |
| Total | 100 |

*Lumateperone equivalent to Lumateperone tosylate

### Brief Manufacturing Process:

a) sucrose, microcrystalline cellulose and talc were co-sifted through appropriate screens;
b) magnesium stearate was separately sifted through appropriate screen;
c) Lumateperone and materials of step a) & some portion of step b) were blended in low shear blender;
d) blended material of step c) were lubricated with the remaining portion of material of step b);
e) the final lubricated blend of step d) were filled into the gelatin based capsule shell of suitable size.

### Example 7

| Ingredients | % w/w |
|---|---|
| Lumateperone* | 15 |
| Sucrose | 68 |
| Lactose anhydrous | 15 |
| Talc | 0.5 |
| Magnesium stearate | 1.5 |
| Total | 100 |

*Lumateperone equivalent to Lumateperone tosylate

### Brief Manufacturing Process:

a) sucrose, lactose anhydrous and talc were co-sifted through appropriate screens;
b) magnesium stearate was separately sifted through appropriate screen;
c) Lumateperone and materials of step a) & some portion of step b) were blended in low shear blender;
d) blended material of step c) were lubricated with the remaining portion of material of step b);
e) the final lubricated blend of step d) were filled into the cellulose based capsule shell of suitable size.

### Example 8

| Ingredients | % w/w |
|---|---|
| Lumateperone* | 20.1 |
| Sucrose | 51.6 |
| Microcrystalline cellulose | 24.5 |
| Croscarmellose sodium | 2.5 |
| Talc | 0.3 |
| Magnesium stearate | 1 |
| Total | 100 |

*Lumateperone equivalent to Lumateperone tosylate

### Brief Manufacturing Process:

a) sucrose, microcrystalline cellulose and talc were co-sifted through appropriate screen and mixed with croscarmellose sodium;
b) magnesium stearate was separately sifted through appropriate screen;
c) Lumateperone and the material obtained from step a) & some portion of step b) were blended in low shear blender;
d) the material obtained in step c) were lubricated in low shear blender with remaining portion of step b);
e) the final lubricated blend of step d) were filled into the cellulose based capsule shell of suitable size.

### Example 9

| Ingredients | % w/w |
|---|---|
| Lumateperone* | 20.1 |
| Microcrystalline cellulose | 78.6 |
| Talc | 0.3 |
| Magnesium stearate | 1 |
| Total | 100 |

*Lumateperone equivalent to Lumateperone tosylate

### Brief Manufacturing Process:

a) microcrystalline cellulose and talc were co-sifted through appropriate screen;
b) magnesium stearate was separately sifted through appropriate screen;
c) Lumateperone and the material obtained from step a) & some portion of step b) were blended in low shear blender;
d) the material obtained in step c) were lubricated in low shear blender with remaining portion of magnesium stearate;
e) the final lubricated blend of step d) were filled into the cellulose based capsule shell of suitable size.

### In-Vitro Dissolution Study:

The release profile of the pharmaceutical composition comprising Lumateperone as per the present specification was evaluated through in-vitro dissolution studies. The compositions were prepared according to the formula and process of example 8-9 and were subjected to an in vitro dissolution study in 500 ml of 0.1N hydrochloric acid at a temperature of 37 ±0.5° C. using a USP apparatus-2 (paddle) with sinker at a rotation of about 50 rpm and results are given in Table-1 below:

TABLE 1
Dissolution profile of commercially marketed Lumateperone
capsules (CAPLYTA ®) and examples 8-9:

| Time point | % Lumateperone released (±5%) | | |
|---|---|---|---|
| (Minutes) | CAPLYTA ® | Example 8 | Example 9 |
| 10 | 82 | 54 | 44.7 |
| 15 | 94 | 91 | 92.7 |
| 20 | 98 | 94 | 96.8 |
| 30 | 98 | 96 | 98.5 |
| 45 | 98 | 97 | 99.5 |
| 60 | 98 | 96 | 99.6 |

### Stability Study:

The stability of the capsule composition comprising Lumateperone as per the present invention was evaluated through accelerated stability studies. The composition was prepared according to the formula and process of example 9, and the composition was subjected to stability study at various temperature and humidity conditions. The composition was found to be stable at accelerated conditions (40° C./75% RH). Table-2 represents the stability study result data.

TABLE 2
stability study result data of example 9:

| | | Lumateperone capsules | | | | |
|---|---|---|---|---|---|---|
| | | | HDPE Bottle Pack | | Blister Pack | |
| Tests | Limit | Initial | 3 M | 6 M | 3 M | 6 M |
| Assay (by | 90-110 | 102.0 | 100.8 | 99.9 | 101.2 | 100.1 |
| HPLC,) | (% w/w) | | | | | |
| Dissolution | NLT 80% | 99 | 97 | 95 | 97 | 92 |
| (by HPLC) | (Q) in | | | | | |
| | 30 mins | | | | | |
| Total degradation | NMT 0.6% | ND | ND | ND | ND | ND |
| Products | w/w | | | | | |

ND: Not determined;
NLT: Not less than;
NMT: Not more than

### Comparative Bioequivalence Study:

“Reference Product” [CAPLYTA® Capsules] Versus “Test Product” [Lumateperone capsules according to present invention].

A randomized, two-treatment, two-period crossover, single-dose oral Bioequivalence study was carried out with Lumateperone capsules 42 mg (Test) according to composition & process of example 9 and compared with CAPLYTA® Capsules 42 mg (Reference) of Intra-Cellular Therapies Inc., USA in healthy, adult, human subjects under fasting and fed condition. T/R (Test/Reference) ratio results are given in table 3 as follows:—

TABLE 3
comparative bioequivalence study results

| | Acceptable | T/R Ratio |
|---|---|---|
| Parameters | limit | (Test - Example 9) |
| Cₘₐₓ/AUCI₀₋ₜ/AUC_{0-inf} -Fasting | 80-125 | Within the |
| | | acceptable limit |
| Cₘₐₓ/AUCI₀₋ₜ/AUC_{0-inf} - Fed | 80-125 | Within the |
| | | acceptable limit |

Based on above T/R ratio, it was found that Test product of example 9 results were well within the acceptable limits and was found to be bio-equivalent to the Reference product.

Therefore, Lumateperone capsules composition which is free of mannitol and disintegrating agent (example-9), gave comparative dissolution, bioavailability profiles and found stable composition with that of CAPLYTA® Capsules.

We claim:

1. A solid oral pharmaceutical composition comprising Lumateperone or pharmaceutically acceptable salt thereof and one or more pharmaceutically acceptable excipients, wherein the composition is free of mannitol and disintegrating agent.

2. The pharmaceutical composition of claim 1, wherein one or more pharmaceutically acceptable excipients are selected from one or more of diluents, lubricants, glidants and mixtures thereof.

3. The pharmaceutical composition of claim 2, wherein the diluent is selected from the group consisting of microcrystalline cellulose, lactose monohydrate, sodium alginate, silicified microcrystalline cellulose, microfine cellulose, dicalcium phosphate, dibasic calcium phosphate, tribasic calcium phosphate, lactose, spray dried lactose, maltodextrin, isomalt, maltitol, lactose anhydrous, sucrose, dextrose, magnesium carbonate and a combination thereof.

4. The pharmaceutical composition of claim 2, wherein the diluent is present in the amount from about 10% to about 90% of the total weight of the composition.

5. The pharmaceutical composition of claim 3, wherein the diluent is selected from sucrose, microcrystalline cellulose or a combination thereof.

6. The pharmaceutical composition of claim 4, wherein the weight ratio of lumateperone to diluent is about 1:2 to about 1:25.

7. The pharmaceutical composition of claim 2, wherein the lubricant is selected from the group consisting of stearic acid, zinc stearate, sodium stearyl fumarate, magnesium stearate, sucrose stearate, aluminum stearate, adipic acid, carnauba wax, glycerol ester of fatty acid, calcium stearate, glycerol tribehenate, glyceryl behenate, polyethylene glycol and a combination thereof.

8. The pharmaceutical composition of claim 2, wherein the glidant is selected from the group consisting of talc, colloidal silica, calcium silicate, magnesium silicate, magnesium trisilicate, fumed silica, bentonite, magnesium carbonate, glyceryl monostearate, glyceryl palmitostearate, light anhydrous silicic acid, crystalline cellulose, calcium phosphate tribasic and a combination thereof.

9. The pharmaceutical composition of claim 1, wherein the composition is in the form of caplets, pills, mini-tablets, granules, pellets, tablets or capsules.

10. The pharmaceutical composition of claim 9, wherein the composition is in the form of capsules.

11. The pharmaceutical composition of claim 10, wherein the capsule shell is made up of hydroxypropyl methylcellulose.

12. A solid oral pharmaceutical composition comprising:
a) from about 1% to about 50% by weight of Lumateperone or its pharmaceutically acceptable salt thereof;
b) from about 20% to about 95% by weight of diluent;
c) from about 0.01% to about 1.5% by weight of glidant;
d) from about 0.1% to about 5% by weight of lubricant;
wherein weight ratio of Lumateperone to diluent is about 1:2 to about 1:10, wherein the composition is free of mannitol and disintegrating agent.

13. The pharmaceutical composition of claim 12, wherein the diluent is selected from the group consisting of microcrystalline cellulose, lactose monohydrate, sodium alginate, silicified microcrystalline cellulose, microfine cellulose, dicalcium phosphate, dibasic calcium phosphate, tribasic calcium phosphate, lactose, spray dried lactose, maltodextrin, isomalt, maltitol, lactose anhydrous, sucrose, dextrose, magnesium carbonate and a combination thereof.

14. The pharmaceutical composition of claim 12, wherein the weight ratio of Lumateperone to diluent is about 1:2 to about 1:25.

15. The pharmaceutical composition of claim 12, wherein the glidant is selected from the group consisting of talc, colloidal silica, calcium silicate, magnesium silicate, magnesium trisilicate, fumed silica, bentonite, magnesium carbonate, glyceryl monostearate, glyceryl palmitostearate, light anhydrous silicic acid, crystalline cellulose, calcium phosphate tribasic and a combination thereof.

16. The pharmaceutical composition of claim 12, wherein the lubricant is selected from the group consisting of stearic acid, zinc stearate, sodium stearyl fumarate, magnesium stearate, sucrose stearate, aluminum stearate, adipic acid, carnauba wax, glycerol ester of fatty acid, calcium stearate, glycerol tribehenate, glyceryl behenate, polyethylene glycol and a combination thereof.

17. A solid oral pharmaceutical composition comprising:
a) from about 1% to about 50% by weight of Lumateperone or its pharmaceutically acceptable salt thereof;
b) from about 20% to about 95% by weight of microcrystalline cellulose;
c) from about 0.01% to about 1.5% by weight of talc;
d) from about 0.1% to about 5% by weight of magnesium stearate;
wherein weight ratio of Lumateperone to microcrystalline cellulose is about 1:2 to about 1:10, wherein the composition is free of mannitol and disintegrating agent and the composition filled into the hydroxypropyl methylcellulose capsule shell.

18. The pharmaceutical composition of claim 12, wherein at least 80% of Lumateperone dissolves from the said composition within 30 minutes in a 500 ml of 0.1N Hydrochloric acid at a temperature of 37 ±0.5° C. using a USP apparatus-2 (paddle) with sinker at a rotation of about 50 rpm.

19. A process for the manufacture of pharmaceutical composition of claim 12 comprising steps of:
a) co-sifting of diluent & glidant through appropriate screen;
b) sifting of lubricant separately through appropriate screen;
c) blending the Lumateperone, material obtained from step a) and some portion material of step b) in low shear blender;
d) lubricating the blended material of step c) with the remaining portion of material of step b);
e) filling the final lubricated blend of step d) into the cellulose capsule shells of suitable size.

20. The composition of claim 1 for treating schizophrenia or depressive episodes associated with bipolar I or II disorder (bipolar depression) in adults.
